(19) United States
(12) Patent Application Publication
(10) Pub. No.: US 2026/0252884 A1
(43) Pub. Date: Aug. 27, 2026

(54) METHOD FOR TRAINING LIGAND INFORMATION GENERATION MODEL, ELECTRONIC DEVICE, AND STORAGE MEDIUM

(71) Applicant: TENCENT TECHNOLOGY (SHENZHEN) COMPANY LIMITED, Shenzhen (CN)

(72) Inventors: Bing HE, Shenzhen (CN); Tianxu LYU, Shenzhen (CN); Jianhua YAO, Shenzhen (CN); Yu ZHAO, Shenzhen (CN); Chenchen QIN, Shenzhen (CN)

(21) Appl. No.: 19/649,843

(22) Filed: Apr. 16, 2026

Related U.S. Application Data
(63) Continuation of application No. PCT/CN2025/077641, filed on Feb. 17, 2025.

(30) Foreign Application Priority Data
Feb. 20, 2024 (CN) 202410192333.2

(51) Int. Cl. G06N 3/08; G16H 70/40

(52) U.S. Cl. CPC G06N 3/08; G16H 70/40

(57) ABSTRACT

A method for training a ligand information generation model performed by an electronic device includes obtaining sample receptor information and sample ligand information, a binding affinity between a ligand described by the sample ligand information and a receptor described by the sample receptor information being not less than a set affinity; denoising reference noise data based on the sample receptor information using a neural network model undergoing training to obtain predicted ligand information; determining a first loss for characterizing a difference between the sample ligand information and the predicted ligand information; and training the neural network model based on the first loss to obtain a ligand information generation model, the ligand information generation model being configured to generate reference ligand information based on reference receptor information.

### CROSS-REFERENCES TO RELATED APPLICATIONS

The present disclosure is a continuation application of PCT Patent Application No. PCT/CN2025/077641, filed on Feb. 17, 2025, which claims priority to Chinese Patent Application No. 202410192333.2, filed on Feb. 20, 2024, all of which is incorporated herein by reference in their entirety.

### FIELD OF THE TECHNOLOGY

Embodiments of the present disclosure relate to the technical field of artificial intelligence, and in particular, to a method and apparatus for training a ligand information generation model, a method and apparatus for generating ligand information, electronic device, and storage medium.

### BACKGROUND OF THE DISCLOSURE

In the drug design industry, ligands may be designed based on artificial intelligence technologies. Ligand-based drugs with therapeutic effects may be obtained through experimental validation of these ligands. Generally, by binding to receptors, ligands trigger signal transduction and biochemical reactions, thereby affecting the receptor function and exerting therapeutic effects. Based on this, the generation of ligands capable of binding to receptors has become a problem requiring urgent resolution.

### SUMMARY

One embodiment of the present disclosure provides a method for training a ligand information generation model, performed by an electronic device. The method includes obtaining sample receptor information and sample ligand information, a binding affinity between a ligand described by the sample ligand information and a receptor described by the sample receptor information being not less than a set affinity; denoising reference noise data based on the sample receptor information using a neural network model undergoing training to obtain predicted ligand information; determining a first loss for characterizing a difference between the sample ligand information and the predicted ligand information; and training the neural network model based on the first loss to obtain a ligand information generation model, the ligand information generation model being configured to generate reference ligand information based on reference receptor information.

Another embodiment of the present disclosure provides an electronic device. The electronic device includes one or more processors and a memory containing at least one computer program that, when being executed, causes the one or more processors to perform: obtaining sample receptor information and sample ligand information, a binding affinity between a ligand described by the sample ligand information and a receptor described by the sample receptor information being not less than a set affinity; denoising reference noise data based on the sample receptor information using a neural network model undergoing training to obtain predicted ligand information; determining a first loss for characterizing a difference between the sample ligand information and the predicted ligand information; and training the neural network model based on the first loss to obtain a ligand information generation model, the ligand information generation model being configured to generate reference ligand information based on reference receptor information.

Another embodiment of the present disclosure provides a non-transitory computer-readable storage medium containing at least one computer program that, when being executed, causes the one or more processors to perform: obtaining sample receptor information and sample ligand information, a binding affinity between a ligand described by the sample ligand information and a receptor described by the sample receptor information being not less than a set affinity; denoising reference noise data based on the sample receptor information using a neural network model undergoing training to obtain predicted ligand information; determining a first loss for characterizing a difference between the sample ligand information and the predicted ligand information; and training the neural network model based on the first loss to obtain a ligand information generation model, the ligand information generation model being configured to generate reference ligand information based on reference receptor information.

### BRIEF DESCRIPTION OF THE DRAWINGS

FIG. 1 is a schematic diagram of an implementation environment of a method for training a ligand information generation model or a method for generating ligand information according to an embodiment of the present disclosure.

FIG. 2 is a flowchart of a method for training a ligand information generation model according to an embodiment of the present disclosure.

FIG. 3 is a schematic diagram of attention processing according to an embodiment of the present disclosure.

FIG. 4 is a schematic diagram of training of a ligand information generation model according to an embodiment of the present disclosure.

FIG. 5 is a flowchart of a method for generating ligand information according to an embodiment of the present disclosure.

FIG. 6 is a schematic diagram of training of a polypeptide sequence denoising diffusion model using a sample target protein as a condition according to an embodiment of the present disclosure.

FIG. 7 is a schematic diagram of comparison between a natural polypeptide and a generated polypeptide according to an embodiment of the present disclosure.

FIG. 8 is another schematic diagram of comparison between a natural polypeptide and a generated polypeptide according to an embodiment of the present disclosure.

FIG. 9 is a schematic diagram of docking score and physicochemical similarity according to an embodiment of the present disclosure.

FIG. 10 is another schematic diagram of comparison between a natural polypeptide and a generated polypeptide according to an embodiment of the present disclosure.

FIG. 11 is a schematic structural diagram of an apparatus for training a ligand information generation model according to an embodiment of the present disclosure.

FIG. 12 is a schematic structural diagram of an apparatus for generating ligand information according to an embodiment of the present disclosure.

FIG. 13 is a schematic structural diagram of a terminal device according to an embodiment of the present disclosure.

FIG. 14 is a schematic structural diagram of a server according to an embodiment of the present disclosure.

### DESCRIPTION OF EMBODIMENTS

To make the objectives, technical solutions, and advantages of the present disclosure clearer, the following further describes implementations of the present disclosure in detail with reference to the accompanying drawings.

In the pharmaceutical design industry, a ligand may be designed based on an artificial intelligence technology, and ligand drugs with therapeutic effects may be obtained through experimental verification of the ligand. For example, the ligand is a polypeptide sequence. The polypeptide sequence is a compound formed by dehydration condensation of at least three amino acids. Generally, the ligand binds to a receptor, thereby affecting the function of the receptor and exerting a therapeutic effect. Based on this, how to generate ligands capable of binding to receptors has become an urgent problem to be resolved.

Embodiments of the present disclosure provide method, apparatus, electronic device, and storage medium for training a ligand information generation model and a method, apparatus, electronic device, and storage medium for generating ligand information. A ligand information generation model can be trained, reference ligand information can be generated based on reference receptor information using the ligand information generation model, and a binding affinity between a ligand described by the reference ligand information and a receptor described by the reference receptor information is relatively high, which is conducive to accelerating the research and development of ligand drugs.

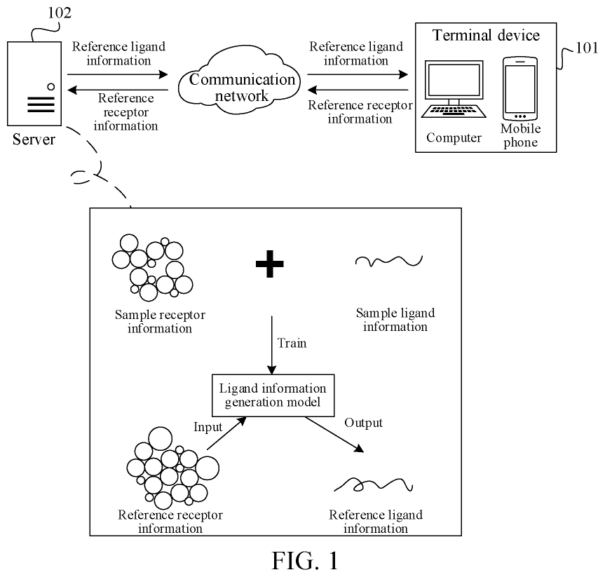

FIG. 1 is a schematic diagram of an implementation environment of a method for training a ligand information generation model or a method for generating ligand information according to an embodiment of the present disclosure. The implementation environment includes a terminal device 101 and a server 102. The method for training a ligand information generation model or the method for generating ligand information in embodiments of the present disclosure may be performed by the terminal device 101, the server 102, or both the terminal device 101 and the server 102.

The terminal device 101 may be a mobile phone or a computer. The mobile phone includes, but is not limited to, a smartphone, a foldable mobile phone, and a flip phone. The computer includes, but is not limited to, a game console, a desktop computer, a tablet computer, and a laptop portable computer. In practical applications, the terminal device 101 may further include a smart television, an intelligent vehicle-mounted device, an intelligent voice interaction device, a smart home appliance, and the like. The server 102 may be one server, a server cluster including a plurality of servers, or either of a cloud computing platform and a virtualization center. This is not limited in the embodiments of the present disclosure. The server 102 may be in communication connection with the terminal device 101 via a communication network, where the communication network is a wired network or a wireless network. The server 102 may have functions of data processing, data storage, data transmission and reception, and the like. This is not limited in the embodiments of the present disclosure. The quantity of terminal devices 101 and servers 102 are not limited, and there may be one or more terminal devices and one or more servers.

In an exemplary embodiment, the terminal device 101 or the server 102 pre-trains a ligand information generation model based on sample receptor information and sample ligand information. When reference receptor information is obtained, the reference receptor information is inputted into the ligand information generation model, and reference ligand information is generated and outputted using the ligand information generation model based on the reference receptor information.

In an exemplary embodiment, the server 102 pre-trains a ligand information generation model based on sample receptor information and sample ligand information. When obtaining reference receptor information, the terminal device 101 transmits the reference receptor information to the server 102 via a communication network. After receiving the reference receptor information, the server 102 inputs the reference receptor information into the ligand information generation model, generates and outputs reference ligand information using the ligand information generation model based on the reference receptor information, and transmits the reference ligand information to the terminal device 101 via the communication network.

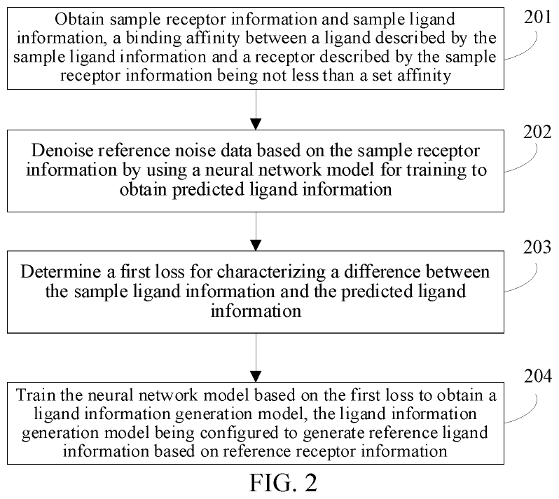

FIG. 2 is a flowchart of a method for training a ligand information generation model according to an embodiment of the present disclosure. For ease of description, the terminal device 101 or the server 102 for performing the method for training a ligand information generation model in embodiments of the present disclosure is referred to as an electronic device. To be specific, the method in the embodiments of the present disclosure may be performed by the electronic device. As shown in FIG. 2, the method includes the following operations.

Operation 201: Obtain sample receptor information and sample ligand information, a binding affinity between a ligand described by the sample ligand information and a receptor described by the sample receptor information being not less than a set affinity.

In the biological field, a receptor is a substance capable of binding to a ligand to thereby induce a biological effect. The biological effect includes anti-oxidation, blood pressure reduction, immune cell activation, and the like. For example, the receptor may be a protein, a nucleic acid sequence, a cell membrane, an antigen, or the like. Generally, the receptor is also referred to as a target. The ligand is a substance capable of binding to the receptor. For example, the ligand may be a protein, a polypeptide sequence, a small molecule, an antibody, or the like. The polypeptide sequence is a substance formed by dehydration condensation of at least three amino acids. The remaining moiety after dehydration condensation of amino acids is referred to as an amino acid residue.

The sample receptor information is configured for describing a sample receptor, and may specifically be feature data for describing the sample receptor. The sample receptor may be any type of receptor. For example, the sample receptor is a protein, and the sample receptor information includes codes, three-dimensional coordinates, and the like of amino acid residues constituting the protein. The sample ligand information is configured for describing a sample ligand, and may specifically be feature data for describing the sample ligand. The sample ligand may be any type of ligand. For example, the sample ligand is a polypeptide sequence, and the sample ligand information includes codes, three-dimensional coordinates, and the like of amino acid residues constituting the polypeptide sequence. The manner of coding amino acid residues is not limited herein. For example, one-hot coding is adopted to code various amino acid residues, so that different amino acid residues correspond to different codes. To be specific, the code of an amino acid residue is unique and is configured for indicating one amino acid residue.

The ligand may bind to the receptor. The strength varies when different ligands bind to different receptors. In the biological field, a binding affinity is used for describing the binding strength between the ligand and the receptor. A higher binding affinity indicates a higher binding strength between the ligand and the receptor. In this example, the binding affinity between the sample ligand and the sample receptor is not less than a set affinity. The binding affinity between the ligand and the receptor is not less than the set affinity, indicating that the binding strength between the ligand and the receptor is relatively high. The binding affinity between the ligand and the receptor is less than the set affinity, indicating that the binding strength between the ligand and the receptor is relatively low. The embodiments of the present disclosure do not limit the determination manner and value of the set affinity. For example, the set affinity is a value determined according to manual experience, or the set affinity is a value obtained through experiments. The experimental process is not repeated herein.

The embodiments of the present disclosure do not limit the manner of obtaining the sample receptor information and the sample ligand information. By way of example, the electronic device may obtain sample receptor information and sample ligand information inputted by a user. Alternatively, the electronic device may acquire a target polypeptide data set from the Internet. The target polypeptide data set includes at least two target polypeptide pairs. One target polypeptide pair includes target information and a polypeptide sequence, and a binding affinity between a target protein described by the target information and a polypeptide described by the polypeptide sequence is not less than a set affinity, where the target information and the polypeptide sequence each include at least two amino acid texts (e.g., amino acid words or amino acid characters). The electronic device may train a feature extraction model, and extract feature data of any target polypeptide pair using the feature extraction model to obtain the sample receptor information and the sample ligand information. The feature extraction model is a network for extracting feature data of amino acid texts, and the structure and feature extraction manner thereof are not limited herein. In this example, the feature data extracted by the feature extraction model is also referred to as embedding. Based on this, the feature extraction model is referred to as an embedding transformation function. To be specific, the electronic device maps each amino acid text in the target information into a corresponding text feature using the embedding transformation function, to obtain the sample receptor information. Similarly, each amino acid text in the polypeptide sequence is mapped into a corresponding text feature using the embedding transformation function, to obtain the sample ligand information.

In some embodiments, for any target polypeptide pair, the target polypeptide pair includes target information x and a polypeptide sequence y. It is assumed that the target information x includes m amino acid texts, where the m amino acid texts are sequentially represented as: x₁, . . . , xₘ. Similarly, the polypeptide sequence y includes n amino acid texts, where the n amino acid texts are sequentially represented as: y₁, . . . y. Then, after mapping the target polypeptide pair using an embedding transformation function EMB(w), sample receptor information EMB(x₁), . . . , EMB(xₘ) and sample ligand information EMB(y₁), . . . , EMB(yₙ) may be obtained. The mapping process may be represented as: EMB(x⊕y)=[EMB(x₁), . . . , EMB(xₘ), EMB(y₁), . . . , EMB(yₙ)], where x⊕y represents concatenation of the polypeptide sequence y following or preceding the target information x, EMB(x₁) represents a text feature of a first amino acid included in the target information x, EMB(xₘ) represents a text feature of an m^{th} amino acid included in the target information x, EMB(yₙ) represents a text feature of a first amino acid included in the polypeptide sequence y, and EMB(yₙ) represents a text feature of an n^{th} amino acid included in the polypeptide sequence y.

The quantity of sample data pairs is at least two. Any two sample data pairs include same or different sample receptor information. Similarly, any two sample data pairs include same or different sample ligand information.

Operation 202: Denoise reference noise data based on the sample receptor information using a to-be-trained neural network model to obtain predicted ligand information.

In the embodiments of the present disclosure, the electronic device may obtain reference noise data for describing a particular type of noise. The noise may be any noise. For example, the noise includes at least one of Gaussian noise, Poisson noise, salt-and-pepper noise, and the like. In some embodiments, the reference noise data is configured for describing noise in which a probability density function obeys a statistical distribution. The statistical distribution is not limited herein. By way of example, the statistical distribution includes at least one of a normal distribution, a U-shaped distribution, a J-shaped distribution, and the like. The probability density function is a commonly used function in statistics and probability theory, and is configured for describing the probability that a continuous random variable is near a particular determined value. Noise in which the probability density function obeys a normal distribution is Gaussian noise, and the reference noise data may be data for describing the Gaussian noise. The embodiments of the present disclosure do not limit the manner of obtaining reference noise data. By way of example, the electronic device may generate reference noise data in any manner, or the electronic device may obtain reference noise data inputted by a user, and the like.

The reference noise data may be inputted into a neural network model. In addition, the sample receptor information may be inputted into the neural network model. In one embodiment, the reference noise data is concatenated preceding or following the sample receptor information to obtain concatenated information, and the concatenated information is inputted into the neural network model. The neural network model is a network model for removing noise data, and is configured to denoise the reference noise data based on the sample receptor information to obtain predicted ligand information. The predicted ligand information includes feature data for characterizing a ligand obtained by prediction through the neural network model.

The embodiments of the present disclosure do not limit a model structure, model parameters, and the like of the neural network model. By way of example, the neural network model includes at least one of a linear layer, a nonlinear layer, an activation layer, an attention layer, a convolution layer, a normalization layer, and the like. For different structures of the neural network model, denoising manners also differ.

In one embodiment, the neural network model includes at least two denoising networks. Operation 202 includes operation 2021 to operation 2023 (not shown in the figure). The at least two denoising networks are connected in series. The denoising network is a network for removing noise data, and the structures, functions, and the like of the denoising networks are similar.

Operation 2021: Denoise, for a first denoising network, the reference noise data based on the sample receptor information using the first denoising network to obtain a denoising result of the first denoising network.

In the embodiments of the present disclosure, the sample receptor information and the reference noise data may be inputted into the first denoising network. Alternatively, the sample receptor information is concatenated preceding or following the reference noise data to obtain concatenated information, and the concatenated information is inputted into the first denoising network. The concatenated information is denoised by the first denoising network to obtain a denoising result of the first denoising network.

The embodiments of the present disclosure do not limit a network structure, model parameters, and the like of the first denoising network. By way of example, the first denoising network includes at least one of a linear layer, a nonlinear layer, an activation layer, an attention layer, a convolution layer, a normalization layer, and the like. For of different structures of the first denoising network, denoising manners also differ. In some embodiments, the first denoising network is a bidirectional encoder representations from transformers (BERT) model or a Transformers network.

In an exemplary embodiment, the first denoising network includes an attention network and a feed-forward network. Operation 2021 includes operation A1 to operation A2 (not shown in the figure). In some embodiments, the attention network includes at least one of a multi-head attention network, a self-attention network, a masked attention network, and the like. The feed-forward network includes at least one of a linear layer, a nonlinear layer, and the like.

Operation A1: Perform attention processing on the sample receptor information and the reference noise data using the attention network to obtain an attention processing result.

In the embodiments of the present disclosure, the sample receptor information and the reference noise data are inputted into the attention network. Alternatively, concatenated information obtained by concatenating the sample receptor information and the reference noise data is inputted into the attention network. Attention processing is performed using the attention network based on an attention mechanism to obtain an attention processing result. Different attention networks correspond to different attention processing manners. A possible attention processing manner is shown below.

By way of example, the sample receptor information includes at least two receptor component information pieces, and the reference noise data includes at least one first sub-data. Operation A1 includes: performing, for any first sub-data, attention processing on the any first sub-data and content information using the attention network to obtain a processing result of the content information, and determining second sub-data corresponding to the any first sub-data based on the processing result of the content information, the content information including the at least two receptor component information pieces and the at least one first sub-data; and determining the attention processing result based on the second sub-data corresponding to the respective first sub-data.

In the embodiments of the present disclosure, the sample receptor information for describing the sample receptor includes at least two receptor component information pieces, where any receptor component information is configured for describing a substance constituting the sample receptor. For example, the sample receptor is a protein, and the protein includes at least one peptide chain. Then any receptor component information may be configured for describing one peptide chain. For example, the receptor component information includes codes and three-dimensional coordinates of amino acid residues in the peptide chain. Alternatively, since the peptide chain includes at least two amino acid residues, any receptor component information may be configured for describing one amino acid residue. For example, the receptor component information includes a code of the amino acid residue.

Similarly, the reference noise data for describing a particular type of noise includes at least one first sub-data. The quantity of such types of noise is at least one, and any first sub-data is configured for describing one noise. Any two first sub-data may describe same or different noises. For example, two first sub-data are different data describing Gaussian noise, or one first sub-data is data describing Gaussian noise and the other first sub-data is data describing salt-and-pepper noise.

In the embodiments of the present disclosure, the respective content information includes at least two receptor component information pieces and at least one first sub-data. In other words, one receptor component information piece may be one piece of content information, and the first sub-data may also include one piece of content information. Therefore, the quantity of pieces of content information is at least two. For any first sub-data, linear mapping or nonlinear mapping is performed on the first sub-data using the attention network to obtain a query (Q) vector corresponding to the first sub-data. For any content information, two different linear mappings or nonlinear mappings are performed on the content information using the attention network, so as to respectively obtain a key (K) vector and a value (V) vector corresponding to the content information.

Next, an attention weight is determined based on the query vector corresponding to the first sub-data and the key vector corresponding to the content information, and the attention weight is multiplied by the value vector corresponding to the content information to obtain a processing result of the content information. In some embodiments, the processing result of the content information is determined according to Formula (1) shown below:

$$A = Attention ( Q , K , V ) = softmax ( \frac{{QK}^{T}}{\sqrt{{d}_{k}}} ) V \qquad Formula ( 1 )$$

A represents the processing result of the content information. Attention represents an attention function used for attention processing, and the function includes three parameters: Q,K,V, where Q represents the query vector corresponding to the first sub-data, K represents the key vector corresponding to the content information, and V represents the value vector corresponding to the content information. softmaxis a normalized exponential function, and T represents a transpose matrix. dₖ represents a dimension of the query vector and the key vector.

$$softmax ( \frac{{QK}^{T}}{\sqrt{{d}_{k}}} )$$

represents the attention weight.

There are at least two pieces of content information, and the processing shown above may be performed on any first sub-data and the respective content information, so as to obtain the processing result of the respective content information. Then, operations such as summation, averaging, and weighting are performed on the processing results of the content information to obtain second sub-data corresponding to the first sub-data.

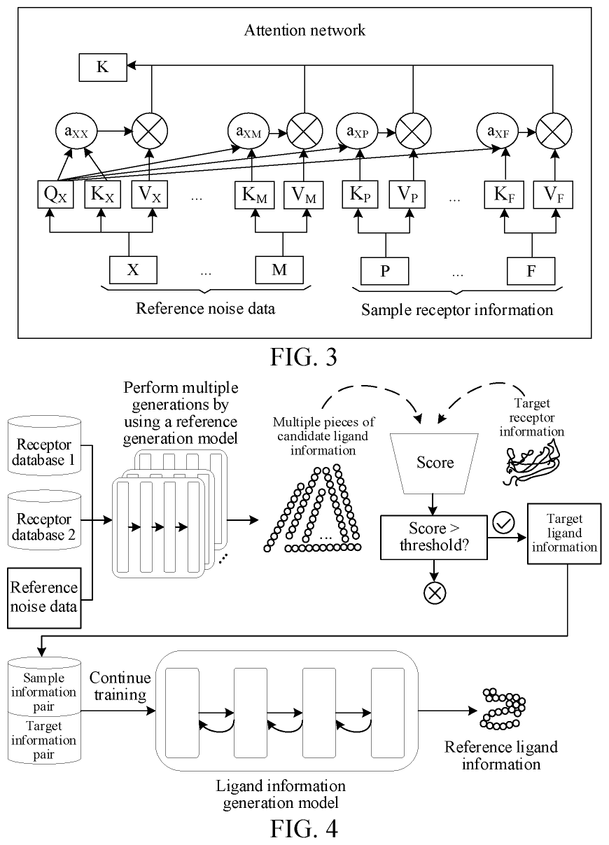

FIG. 3 is a schematic diagram of attention processing according to an embodiment of the present disclosure. The reference noise data includes first sub-data X to first sub-data M, and the sample receptor information includes receptor component information P to receptor component information F. A query vector Q_{X} of first sub-data X is obtained by mapping first sub-data X through an attention network. For each piece of content information i (where i takes any value from X to M and from P to F) in first sub-data X to first sub-data M and receptor component information P to receptor component information F, a key vector Kᵢ and a value vector Vᵢ of content information i are obtained by mapping content information i through the attention network. An attention weight a_{Xi} is determined based on the query vector Q_{X} of first sub-data X and the key vector Kᵢ of content information i, and the attention weight a_{Xi} is multiplied by the value vector Vᵢ of content information i to obtain a processing result of content information i. The processing results of the respective content information are averaged to obtain second sub-data K corresponding to first sub-data X.

In a similar processing manner, second sub-data corresponding to the respective first sub-data except first sub-data X may be obtained. Details are not repeated herein. Then, the second sub-data corresponding to the respective first sub-data are concatenated to obtain an attention processing result.

On the one hand, the second sub-data corresponding to any first sub-data is obtained by performing attention calculation on the first sub-data and the respective receptor component information. Therefore, the second sub-data is obtained by processing the first sub-data with reference to the respective receptor component information, which is equivalent to denoising the first sub-data under the guidance of the sample receptor information to obtain the second sub-data. By removing noise data in the first sub-data under the guidance of the sample receptor information, the ability of the second sub-data to characterize ligand component information is improved. By concatenating the respective second sub-data to obtain an attention processing result, the ability of the attention processing result to characterize a ligand having the characteristic of binding to the sample receptor is improved.

On the other hand, the second sub-data corresponding to any first sub-data is obtained by performing attention calculation on the first sub-data and the respective first sub-data. Therefore, the second sub-data is obtained by processing the first sub-data with reference to the respective first sub-data, thereby improving the correlation between the second sub-data and the reference noise data, enabling the second sub-data to have correlation with each other, and thus improving the accuracy of the attention processing result to characterize the ligand.

The foregoing manner of determining the attention processing result is merely exemplary, and in practical applications, there may be other determination manners. For example, on the one hand, a query vector is obtained by mapping the reference noise data through the attention network. On the other hand, two different mappings are performed on the sample receptor information through the attention network to obtain a key vector and a value vector respectively. An attention weight is determined based on the query vector and the key vector, and the attention weight is multiplied by the value vector to obtain the attention processing result.

Operation A2: Determine a denoising result of the first denoising network based on the attention processing result using the feed-forward network.

In the embodiments of the present disclosure, the attention processing result may be inputted into a feed-forward network, and the attention processing result is processed by the feed-forward network to obtain a processing result of the feed-forward network. The processing result of the feed-forward network is used as the denoising result of the first denoising network, or processing such as mapping, activation, or normalization is performed on the processing result of the feed-forward network to obtain the denoising result of the first denoising network.

In an exemplary embodiment, the feed-forward network includes at least one of a linear layer, a nonlinear layer, and the like, and at least one processing such as linear mapping or nonlinear mapping may be performed on the attention processing result using the feed-forward network to obtain the processing result of the feed-forward network.

In some embodiments, operation A2 includes: performing mapping on the attention processing result using the feed-forward network to obtain a mapping result; and performing mapping on the mapping result or a set feature using the feed-forward network to obtain the denoising result of the first denoising network.

In the embodiments of the present disclosure, the feed-forward network includes a first mapping layer, a selection layer, and a second mapping layer. The first mapping layer may be a linear layer or a nonlinear layer. Linear mapping or nonlinear mapping is performed on the attention processing result using the first mapping layer to obtain a mapping result. An extreme value is selected from the mapping result and a set feature using the selection layer. The extreme value herein is a maximum value or a minimum value, and is also the mapping result or the set feature. The second mapping layer may be a linear layer or a nonlinear layer. Linear mapping or nonlinear mapping is performed on the extreme value using the second mapping layer to obtain a processing result of the feed-forward network. Then, the denoising result of the first denoising network is determined based on the processing result of the feed-forward network.

The set feature is a preset feature, and the feature may be a number, a matrix, or the like. By way of example, the processing result of the feed-forward network is determined according to Formula (2) shown below. In Formula (2), the set feature is number 0.

$$FN ( A ) = max ( 0 , {AW}_{1} + {b}_{1} ) {W}_{2} + {b}_{2} \qquad Formula ( 2 )$$

FFN(A) represents the processing result of the feed-forward network. max represents a maximum value function used by the selection layer. AW₁+b₁ represents the mapping result obtained by the first mapping layer, where A represents the attention processing result, W₁ represents a weight parameter in a mapping function used by the first mapping layer, and b₁ represents a bias parameter in the mapping function used by the first mapping layer. W₂ represents a weight parameter in a mapping function used by the second mapping layer, and b₂ represents a bias parameter in the mapping function used by the second mapping layer.

Data involved in denoising performed by the first denoising network is data at a feature level. In other words, the sample receptor information, the reference noise data, the denoising result, and the like are all data at the feature level.

In some embodiments, the neural network model further includes a first embedding layer, and the first embedding layer precedes the first denoising network by series connection. The electronic device may obtain first media information for describing the sample receptor and second media information for describing the sample ligand. The first media information or the second media information includes at least one of text, audio, image, video, and the like. For example, when the sample receptor is a protein, the first media information may be a visualized image of the protein. For another example, when the sample ligand is a polypeptide, the second media information may include types of amino acids included in the polypeptide, as well as a quantity and three-dimensional coordinates of each type of amino acid. The first media information and the second media information are both media information, and the media information includes at least two information segments. For example, when the media information is text, the media information includes at least two characters. When the media information is audio, the media information includes at least two audio frames. When the media information is an image, the media information includes at least two pixel values. When the media information is a video, the media information includes at least two frame images.

The respective information segments included in the first media information may be mapped into corresponding embedding features using the first embedding layer to obtain the sample receptor information. The respective information segments included in the second media information are mapped into corresponding embedding features using the first embedding layer to obtain the sample ligand information. Next, the reference noise data is denoised based on the sample receptor information using the first denoising network to obtain the denoising result of the first denoising network.

Operation 2022: Denoise, for a non-first denoising network, a denoising result of a previous denoising network of the non-first denoising network based on the sample receptor information using the non-first denoising network to obtain a denoising result of the non-first denoising network.

In the embodiments of the present disclosure, the denoising result of the first denoising network may be inputted into a second denoising network. The denoising result of the first denoising network is denoised using the second denoising network based on the sample receptor information according to a denoising principle of the first denoising network, to obtain a denoising result of the second denoising network. The denoising process is not repeated herein. Next, the denoising result of the second denoising network may be inputted into a third denoising network. The denoising result of the second denoising network is denoised using the third denoising network based on the sample receptor information according to the denoising principle of the first denoising network, to obtain a denoising result of the third denoising network. The denoising process is not repeated herein. By analogy according to the foregoing denoising principle, the denoising result of the last denoising network is obtained.

It can be seen from the content of operation 2021 to operation 2022 that, in some embodiments, the reference noise data is gradually denoised by constructing at least two denoising networks connected in series until the denoising result of the last denoising network is obtained. The denoising process may be represented as Formula (3) shown below.

$${p}_{θ} = ( {l}_{t - 1} \vert {l}_{t} ) = N ( {l}_{t - 1} ; {μ}_{θ} ) ( {l}_{t} , t ) , {Σ}_{θ} ( {l}_{t} , t ) ) \qquad Formula ( 3 )$$

For a particular denoising network, lₜ represents an input of the denoising network, namely the reference noise data mentioned above or the denoising result of a previous denoising network, and t represents the number of denoising operations corresponding to the denoising network. lₜ₋₁ represents an output of the denoising network, namely the denoising result of the denoising network mentioned above. p_{θ}(lₜ₋₁|lₜ) represents a distribution satisfied by the output lₜ₋₁ obtained by denoising the input lₜ through the denoising network, where θ represents a network parameter of the denoising network. N represents a symbol of Gaussian distribution. μ_{θ}(Iᵢt) represents a mean value of the Gaussian distribution, and Σ_{θ}(l₁,t) represents a variance of the Gaussian distribution.

Operation 2023: Determine the predicted ligand information based on a denoising result of a last denoising network.

In the embodiments of the present disclosure, the denoising result of the last denoising network may be used as the predicted ligand information, or the predicted ligand information may be determined based on the denoising result of the last denoising network by means of linear mapping, nonlinear mapping, or the like. The predicted ligand information is data at a feature level, and is configured for characterizing a ligand capable of binding to the receptor described by the sample receptor information. The ligand herein is a ligand of the same type as the sample ligand. For example, when the sample ligand is a polypeptide sequence, the ligand characterized by the predicted ligand information is also a polypeptide sequence. In some embodiments, the predicted ligand information includes codes, three-dimensional coordinates, and the like of amino acids constituting the polypeptide sequence.

In an exemplary embodiment, the denoising result of the last denoising network includes at least two predicted sub-features. Operation 2023 includes: screening, for any predicted sub-feature, at least two target ligand component information pieces from at least two candidate ligand component information pieces based on feature similarity between the any predicted sub-feature and the at least two candidate ligand component information pieces, and sampling from the at least two target ligand component information pieces to obtain ligand component information corresponding to the any predicted sub-feature; and determining the predicted ligand information based on the ligand component information corresponding to the respective predicted sub-features.

In the embodiments of the present disclosure, the reference noise data includes at least one first sub-data. For any first sub-data, after denoising the first sub-data by the respective denoising networks in sequence according to the manners of operation A1 and operation A2, a predicted sub-feature outputted by the last denoising network is obtained. The predicted sub-feature is data at a feature level, and is feature data of a substance constituting a ligand, for describing the substance. For example, the ligand is a protein, and the predicted sub-feature may be a feature for describing a peptide chain or an amino acid residue.

The electronic device may obtain a dictionary, and the dictionary includes at least two candidate ligand component information pieces. Any candidate ligand component information is feature data of a substance constituting a ligand, for describing the substance. For example, the ligand is a protein, and the protein is composed of a plurality of amino acid residues. The candidate ligand component information may be configured for describing an amino acid residue such as a glycine residue, an alanine residue, or a tryptophan residue.

For any predicted sub-feature, a feature similarity may be calculated according to a similarity calculation formula based on the predicted sub-feature and any candidate ligand component information. The embodiments of the present disclosure do not limit the similarity calculation formula. By way of example, the similarity calculation formula includes any one of a cosine similarity calculation formula, a Euclidean distance calculation formula, a Manhattan distance calculation formula, and the like. According to the manner, the feature similarity between any predicted sub-feature and at least two candidate ligand component information pieces may be calculated.

Next, target ligand component information having a feature similarity not less than a similarity threshold is screened from the at least two candidate ligand component information pieces. In other words, the target ligand component information is candidate ligand component information having a feature similarity with the predicted sub-feature not less than the similarity threshold. The embodiments of the present disclosure do not limit the similarity threshold. By way of example, the similarity threshold is a value set according to manual experience. Alternatively, the similarity threshold is any feature similarity among feature similarities between any predicted sub-feature and at least two candidate ligand component information pieces. For example, the feature similarities between any predicted sub-feature and at least two candidate ligand component information pieces are sorted, and an Nth (N is a positive integer) feature similarity after sorting is used as the similarity threshold.

There are at least two pieces of target ligand component information. Ligand component information corresponding to any predicted sub-feature may be obtained by random sampling from the at least two pieces of target ligand component information, or by sampling from the at least two pieces of target ligand component information based on feature similarities corresponding to the respective target ligand component information. The feature similarities corresponding to any two pieces of target ligand component information may be the same or different. In a sampling process, the feature similarity corresponding to the target ligand component information may represent a probability of sampling the target ligand component information. For example, if the feature similarity corresponding to the target ligand component information is 95%, the target ligand component information has a 95% probability of being sampled and a 5% probability of not being sampled.

According to the foregoing manner, ligand component information corresponding to the respective predicted sub-features may be obtained by sampling. The ligand component information corresponding to the respective predicted sub-features is concatenated to obtain concatenated information, the concatenated information is predicted ligand information, and a ligand is characterized by the predicted ligand information. Alternatively, randomness in sampling the ligand component information causes the concatenated information to carry noise. Based on this, the concatenated information may be denoised using a target denoising network to obtain predicted ligand information. The target denoising network is a network trained based on massive ligand information and having a function of denoising ligand information, and has learned sequence features of the ligand information. A network structure and a denoising manner are not limited herein. The massive ligand information means that the quantity of ligand information is not less than a quantity threshold. The quantity threshold may be set according to manual experience. For example, the quantity threshold is 500. If the concatenated information carries noise, the concatenated information may be regarded as ligand information carrying noise. The ligand information carrying noise is denoised using the target denoising network to obtain denoised ligand information, so as to fine-tune the concatenated information and obtain predicted ligand information with higher accuracy. The denoised ligand information is the predicted ligand information.

In some embodiments, the concatenated information includes at least two pieces of concatenated sub-information. For any concatenated sub-information, attention processing is performed on any concatenated sub-information and the respective concatenated sub-information using the target denoising network to obtain a processing result of the respective concatenated sub-information, and a denoising result corresponding to any concatenated sub-information is determined based on the processing result of the respective concatenated sub-information. The predicted ligand information is determined based on the denoising result corresponding to the respective concatenated sub-information. For the implementation, refer to the description of operation A1. Implementation principles thereof are similar. Details are not repeated herein.

In practical applications, the model may output at least one piece of predicted ligand information based on the foregoing implementation principle. In the embodiments of the present disclosure, on the one hand, accuracy of ligand component information predicted by the model is ensured by screening target ligand component information from candidate ligand component information. On the other hand, diversity of the ligand component information predicted by the model is ensured by sampling, from the target ligand component information, ligand component information corresponding to the predicted sub-features. Based on this, it is ensured that the model can predict predicted ligand information with high accuracy, and diversity of the predicted ligand information is ensured.

In some embodiments, the neural network model further includes a second embedding layer, and the second embedding layer follows the last denoising network by series connection. The predicted ligand information includes at least two features (i.e., ligand component information), and the respective features are mapped into corresponding information segments using the second embedding layer to obtain third media information for characterizing a ligand.

Operation 203: Determine a first loss for characterizing a difference between the sample ligand information and the predicted ligand information.

In the embodiments of the present disclosure, a first loss may be calculated according to a calculation formula of a first loss function based on the sample ligand information and the predicted ligand information. The embodiments of the present disclosure do not limit the first loss function. By way of example, the first loss function is any one of a mean square error loss function, a cross-entropy loss function, a relative entropy loss function, and the like. In some embodiments, the first loss is calculated according to Formula (4) shown below:

$$ℒ = \frac{1}{n} \sum {( y - \hat{y} )}^{2} \qquad Formula ( 4 )$$

L represents the first loss. n represents the quantity of sample ligand information, and also represents the quantity of predicted ligand information. y represents the sample ligand information, and ŷ represents the predicted ligand information.

Operation 204: Train the neural network model based on the first loss to obtain a ligand information generation model, the ligand information generation model being configured to generate reference ligand information based on reference receptor information.

In the embodiments of the present disclosure, a gradient of the first loss with respect to model parameters of the neural network model may be calculated, and the model parameters of the neural network model are adjusted based on the gradient, so as to implement one training on the neural network model and obtain a trained neural network model.

If the trained neural network model satisfies a training end condition, the trained neural network model is used as a ligand information generation model. If the trained neural network model does not satisfy the training end condition, the trained neural network model is used as a neural network model for next training, and the neural network model is trained next time according to the manners of operation 201 to operation 204 to obtain a trained neural network model, and the ligand information generation model is determined based on the trained neural network model.

The embodiments of the present disclosure do not limit the condition for the trained neural network model to satisfy the training end condition. By way of example, the condition that the trained neural network model satisfies the training end condition includes: the number of training operations of the trained neural network model reaches a set number, or an error of the trained neural network model is within a set error range, and the like. Both the set number and the set error range may be set according to manual experience.

The first loss is configured for characterizing a difference between the sample ligand information and the predicted ligand information. Training the neural network model using the first loss may enable the neural network model to adjust in a direction of making the outputted predicted ligand information close to the sample ligand information, and improve accuracy of the predicted ligand information outputted by the neural network model. In addition, since a binding affinity between a ligand described by the sample ligand information and a receptor described by the sample receptor information is not less than a preset affinity, and the predicted ligand information continuously approaches the sample ligand information in a process of training the ligand information generation model, the ligand information generation model can generate reference ligand information for describing the ligand having a high binding affinity with the receptor.

As mentioned above, the embodiments of the present disclosure do not limit the manner of obtaining the reference noise data. An exemplary embodiment is shown below. In this implementation, operation 205 (not shown in the figure) is further included before operation 202.

Operation 205: Add noise to the sample ligand information to obtain the reference noise data.

The embodiments of the present disclosure do not limit the manner of adding noise to the sample ligand information. In some embodiments, noise is added to the sample ligand information based on a noise function of at least one noise such as Gaussian noise or Poisson noise, to obtain the reference noise data.

In an exemplary embodiment, noise addition is performed at least twice. Operation 205 includes operation B1 to operation B2 (not shown in the figure).

Operation B1: Perform, for a first noise addition, the first noise addition on the sample ligand information to obtain a noise addition result of the first noise addition.

In the embodiments of the present disclosure, noise data for a first noise addition may be generated in any manner, and fusion calculation is performed on the noise data for the first noise addition and the sample ligand information, so as to implement adding the noise data for the first noise addition to the sample ligand information and obtain a noise addition result of the first noise addition.

Operation B2: Perform, for a non-first noise addition, the non-first noise addition on a noise addition result of a previous noise addition of the non-first noise addition to obtain a noise addition result of the non-first noise addition, a noise addition result of a last noise addition being the reference noise data.

In the embodiments of the present disclosure, noise data for a second noise addition may be generated in any manner, and fusion calculation is performed on the noise data for the second noise addition and the noise addition result of the first noise addition, so as to implement adding the noise data for the second noise addition to the noise addition result of the first noise addition and obtain a noise addition result of the second noise addition.

Next, noise data for a third noise addition may be generated in any manner, and fusion calculation is performed on the noise data for the third noise addition and the noise addition result of the second noise addition, so as to implement adding the noise data for the third noise addition to the noise addition result of the second noise addition and obtain a noise addition result of the third noise addition.

By analogy according to the foregoing noise addition principle, at least two noise additions are sequentially performed on the sample ligand information to obtain a noise addition result of a last noise addition, where the noise addition result is the reference noise data. In some embodiments, the noise addition process may be represented as Formula (5) shown below:

$$q ( {l}_{t} \vert {l}_{t - 1} ) = 𝒩 ( {l}_{t} ; \sqrt{1 - {β}_{t}} {l}_{t - 1} , {β}_{t} l ) \qquad Formula ( 5 )$$

lₜ represents data obtained after noise addition, namely the noise addition result of the first noise addition or the noise addition result of the non-first noise addition mentioned above, which may be described as a noise addition result of a t^{th} noise addition. lₜ₋₁ represents data before noise addition, namely the sample ligand information mentioned above or the noise addition result of a previous noise addition of the non-first noise addition, which may be described as the sample ligand information or a noise addition result of a (t−1)^{th} noise addition. q(lₜ|lₜ₋₁) represents a distribution satisfied by lₜ obtained after noise addition on iₜ₋₁. N represents a symbol of Gaussian distribution. \sqrt{1−βₜlₜ₋₁} represents a mean value of the Gaussian distribution, and βₜI represents a variance of the Gaussian distribution. β_{t′}I are two hyperparameters.

In one embodiment, the noise addition and the denoising are both performed T times, T being a positive integer. Operation 204 includes: determining, for an i^{th} denoising, a second loss corresponding to the i^{th} denoising based on noise data removed during the i^{th} denoising and noise data added during a (T+1−i)^{th} noise addition, i being a positive integer from 1 to T; and training the neural network model based on the first loss and the second loss corresponding to each denoising to obtain the ligand information generation model.

In the embodiments of the present disclosure, the reference noise data is obtained by gradually performing noise addition T times on the sample ligand information, and the predicted ligand information is obtained by gradually performing denoising T times on the reference noise data. The noise addition process and the denoising process are inverse processes of each other. The first noise addition corresponds to a T^{th} denoising, the second noise addition corresponds to a (T−1)th denoising, and so on, and a T^{th} noise addition corresponds to the first denoising. Based on this, an i^{th} denoising corresponds to a (T+1−i)^{th} noise addition.

As mentioned above, the electronic device may generate noise data for the (T+1−i)^{th} noise addition in any manner. In addition, the electronic device may further determine noise data for the i^{th} denoising based on an input and an output of an i^{th} denoising network, for example, reference noise data inputted by the first denoising network and a denoising result outputted by the first denoising network, or a denoising result of a previous denoising network inputted by a non-first denoising network and a denoising result outputted by the non-first denoising network. The determination manner is not repeated herein. For example, the noise data for the i^{th} denoising is obtained by subtracting the input from the output of the i^{th} denoising network, or the noise data for the i^{th} denoising is determined based on a distribution satisfied by the output of the i^{th} denoising network and a distribution satisfied by the input of the i^{th} denoising network.

Next, a second loss corresponding to the i^{th} denoising may be calculated according to a calculation formula of a second loss function based on the noise data for the (T+1−i)^{th} noise addition and the noise data for the i^{th} denoising. The embodiments of the present disclosure do not limit the second loss function. By way of example, the second loss function is any one of a mean square error loss function, a cross-entropy loss function, a relative entropy loss function, and the like.

Then, a loss of the neural network model may be determined based on the first loss and the second losses corresponding to the respective denoising. For example, operations such as weighted summation and weighted averaging are performed on the first loss and the second losses corresponding to the respective denoising to obtain the loss of the neural network model. Next, a gradient of the loss of the neural network model with respect to model parameters of the neural network model is calculated, and the model parameters of the neural network model are adjusted based on the gradient to obtain the ligand information generation model. For the adjustment process, refer to the description of operation 204. Details are not repeated herein.

Adjusting the model parameters of the neural network model using the second losses corresponding to the respective denoising may optimize the neural network model in a direction of making denoised data close to noise addition data, thereby optimizing the neural network model in a direction of making the outputted predicted ligand information close to the sample ligand information. Through the foregoing optimization, the ligand information generation model can relatively accurately restore the sample ligand information by denoising, thereby improving the accuracy of the ligand information generation model.

In an exemplary embodiment, operation 204 includes operation 2041 to operation 2043 (not shown in the figure).

Operation 2041: Train the neural network model based on the first loss to obtain a reference generation model.

In the embodiments of the present disclosure, a gradient of the first loss with respect to model parameters of the neural network model may be calculated, and the model parameters of the neural network model are adjusted based on the gradient, so as to implement one training on the neural network model and obtain a trained neural network model.

If the trained neural network model satisfies a training end condition, the trained neural network model is used as a reference generation model. If the trained neural network model does not satisfy the training end condition, the trained neural network model is used as a neural network model for next training, and the neural network model is trained next time according to the manners of operation 201 to operation 204 to obtain a trained neural network model, and the reference generation model is determined based on the trained neural network model.

Operation 2042: Denoise the reference noise data based on target receptor information using the reference generation model to obtain target ligand information.

In the embodiments of the present disclosure, the electronic device may obtain target receptor information, and the obtaining manner is not limited herein. By way of example, the electronic device may obtain target receptor information inputted by a user. Alternatively, the electronic device may obtain a receptor data set. For example, the receptor data set is a Swiss-Prot data set or a UniRef30 data set. Bot the data sets are data sets related to proteins.

Receptor data may be selected from the receptor data set, and target receptor information may be obtained by mapping the receptor data into corresponding features. For example, protein data with a sequence length of less than 1000 amino acids is extracted from the Swiss-Prot data set or the UniRef30 data set, and the respective amino acid data in the protein data is mapped into corresponding features to obtain a protein feature, thereby obtaining target receptor information. The target receptor information is feature data for describing a target receptor. The target receptor is any receptor in which the quantity of receptor component information is lower than a threshold, where the threshold may be set according to manual experience. For example, in the foregoing example, the threshold is 1000.

Since the reference generation model is obtained based on training of the neural network model, the implementation principle of the reference generation model is similar to that of the neural network model. Based on this, the reference noise data and the target receptor information may be inputted into the reference generation model. The reference generation model denoises the reference noise data based on the target receptor information according to the implementation manner described in operation 202 to obtain target ligand information. The denoising manner is not repeated herein.

In one embodiment, operation 2042 includes: denoising the reference noise data based on target receptor information using the reference generation model to obtain candidate ligand information; and determining, if a first quality indicator for characterizing quality of the candidate ligand information satisfies a first indicator condition, the candidate ligand information as the target ligand information.

In the embodiments of the present disclosure, the electronic device may denoise the reference noise data based on the target receptor information using the reference generation model according to the implementation principles of operation 2021 to operation 2023, so as to obtain candidate ligand information. The denoising process is not repeated herein. The reference generation model may generate at least two pieces of candidate ligand information. The candidate ligand information is not only high in accuracy but also diverse.

For any candidate ligand information, the electronic device may determine a first quality indicator of the candidate ligand information, to describe the quality of the candidate ligand information using the first quality indicator of the candidate ligand information. Generally, a larger first quality indicator of the candidate ligand information indicates a higher quality of the candidate ligand information. The embodiments of the present disclosure do not limit the manner of determining the first quality indicator of the candidate ligand information. By way of example, the first quality indicator may be determined according to any one example of implementation A to implementation C shown below.

In implementation A, at least two pieces of first conformation information are determined based on the target receptor information and the candidate ligand information, the first conformation information being configured for describing a complex conformation formed after binding a receptor described by the target receptor information and a ligand described by the candidate ligand information. For any first conformation information, a first indicator of the any first conformation information is determined, the first indicator being configured for characterizing binding energy of a complex conformation described by the any first conformation information. A first quality indicator of the candidate ligand information is determined based on the first indicator of the respective first conformation information.

In the embodiments of the present disclosure, the electronic device may determine at least two pieces of binding information of the candidate ligand information based on any one of search algorithms such as a Monte Carlo simulation algorithm, a genetic algorithm, and a particle swarm optimization algorithm. The binding information of the candidate ligand information is configured for describing at least one piece of information such as a posture, a position, a rotation, and a translation of the ligand when the ligand described by the candidate ligand information binds to the receptor described by the target receptor information.

Next, first conformation information is determined based on the target receptor information and any binding information of the candidate ligand information, so as to implement transformation on the ligand in aspects of the posture, position, rotation, and translation based on the binding information of the candidate ligand information, bind the receptor to the transformed ligand to obtain a complex conformation, and describe the complex conformation using the first conformation information. The complex conformation refers to a structure formed by binding the ligand to the receptor.

Then, for any first conformation information, the electronic device may calculate a first indicator of the first conformation information using an indicator function such as a physical function or a potential energy function. The indicator function is configured for calculating binding energy of the complex conformation based on van der Waals forces between molecules in the complex conformation, chemical bonds between atoms, and the like. Based on this, the first indicator of the first conformation information may represent binding energy of the complex conformation described by the first conformation information.

In the foregoing manner, the electronic device may determine respective first conformation information based on the target receptor information and the respective binding information of the candidate ligand information, and calculate first indicators of the respective first conformation information. Next, a median or a mean value of the first indicator is determined based on the first indicators of the respective first conformation information, so as to obtain a first quality indicator of the candidate ligand information.

In implementation B, reference conformation information is selected from a database, an alignment result between ligand information included in the reference conformation information and the candidate ligand information satisfying an alignment condition, and an alignment result between receptor information included in the reference conformation information and the target receptor information satisfying the alignment condition. Second conformation information is determined based on the reference conformation information, the target receptor information, and the candidate ligand information, the second conformation information being configured for describing a complex conformation formed after binding a receptor described by the target receptor information and a ligand described by the candidate ligand information. A first quality indicator of the candidate ligand information is determined based on the second conformation information.

In the embodiments of the present disclosure, the electronic device may obtain a database, where the database includes at least two pieces of candidate conformation information. Any candidate conformation information includes ligand information and receptor information, and the candidate conformation information is configured for describing a complex conformation formed by binding a ligand described by the ligand information to a receptor described by the receptor information, where the complex conformation is an actually existing complex conformation. Different candidate conformation information describes different complex conformations. Since the ligand may bind to the receptor by performing transformation in aspects of a posture, a position, a rotation, and a translation, and the receptor may correspond to different postures, positions, rotations, and translations, the same ligand and receptor may be bound to obtain at least one complex conformation. Based on this, any two different complex conformations may correspond to the same or different ligands and the same or different receptors.

For any candidate conformation information, the electronic device may determine a first alignment result based on the candidate ligand information and the ligand information included in the candidate conformation information, where the first alignment result characterizes an alignment degree between the candidate ligand information and the ligand information included in the candidate conformation information. The ligand described by the candidate ligand information and the ligand described by the ligand information included in the candidate conformation information may be aligned first, so that arrangements of the two ligands are as consistent as possible. Since the ligand includes at least two substances constituting the ligand, the two aligned ligands may be regarded as substances in two rows and at least two columns. Similarities and differences between the two rows of substances are compared column by column, and the alignment degree between the candidate ligand information and the ligand information included in the candidate conformation information is determined based on a comparison result of each column. For example, the ligand is a protein including at least two amino acid residues, and aligning the two proteins means making arrangements of amino acid residues of the two proteins as consistent as possible. The aligned ligand is regarded as one row of ligands, and one amino acid residue in the row of ligands may be regarded as being located in a particular column in the row of ligands. In other words, the two rows of aligned ligands may be regarded as amino acid residues in two rows and multiple columns. Similarities and differences between the two rows of amino acid residues are compared column by column, and the alignment degree between the two proteins is determined based on a comparison result of each column.

In some embodiments, the first alignment result is positively correlated with the alignment degree. A larger first alignment result indicates a higher alignment degree. If the first alignment result is not less than an alignment threshold, the first alignment result satisfies the alignment condition. If the first alignment result is less than the alignment threshold, the first alignment result does not satisfy the alignment condition.

Similarly, for any candidate conformation information, the electronic device may determine a second alignment result based on the target receptor information and the receptor information included in the candidate conformation information, where the second alignment result characterizes an alignment degree between the target receptor information and the receptor information included in the candidate conformation information. In some embodiments, the second alignment result is positively correlated with the alignment degree. A larger second alignment result indicates a higher alignment degree. If the second alignment result is not less than an alignment threshold, the second alignment result satisfies the alignment condition. If the second alignment result is less than the alignment threshold, the second alignment result does not satisfy the alignment condition.

The embodiments of the present disclosure do not limit the manner of determining the alignment threshold. By way of example, the alignment threshold is a value set according to manual experience, or a value adaptively adjusted according to actual situations. For example, when there are a plurality of alignment results higher than 0.5, the alignment threshold may be increased accordingly, and when there are few alignment results higher than 0.5, the alignment threshold may be decreased accordingly.

If the first alignment result of any candidate conformation information satisfies the alignment condition and the second alignment result of the candidate conformation information satisfies the alignment condition, the candidate conformation information is determined as the reference conformation information. In this manner, at least one piece of reference conformation information may be determined.

Next, the electronic device may determine first distribution information and second distribution information based on the reference conformation information, the target receptor information, and the candidate ligand information. The electronic device learns some properties of the reference conformation information, such as a posture, rotation, translation, and position of the ligand described by the reference conformation information, and a posture, rotation, translation, and position of the receptor described by the reference conformation information, and determines the first distribution information and the second distribution information based on the information, the target receptor information, and the candidate ligand information. The first distribution information is configured for describing a distribution satisfied by a distance between adjacent constituent substances in the receptor described by the target receptor information and a distribution satisfied by an included angle formed by the adjacent constituent substances (for example, when the receptor is a protein, the constituent substances are amino acids or peptide chains), and the posture, rotation, translation, and position of the receptor described by the target receptor information is reflected using the distributions. Similarly, the second distribution information is configured for describing a distribution satisfied by a distance between adjacent constituent substances in the ligand described by the candidate ligand information (for example, when the receptor is a protein, the constituent substances are amino acids or peptide chains) and a distribution satisfied by an included angle formed by the adjacent constituent substances, and the posture, rotation, translation, and position of the ligand described by the candidate ligand information is reflected using the distributions.

Then, the electronic device determines second conformation information based on the first distribution information and the second distribution information, where the second conformation information is configured for describing a complex conformation formed after binding a receptor described by the target receptor information and a ligand described by the candidate ligand information.

A similarity degree between the target receptor information and the receptor information corresponding to the reference conformation information is relatively high, and a similarity degree between the candidate ligand information and the ligand information corresponding to the reference conformation information is relatively high. Generally, a higher similarity of the ligand information indicates a closer folding manner, and a higher similarity of the receptor information indicates a closer folding manner. Based on this, determining the second conformation information based on the reference conformation information may make the complex conformation described by the second conformation information closer to an actual situation, thereby improving the accuracy of the second conformation information.

Then, the electronic device may calculate a second indicator of the second conformation information using an indicator function such as a physical function or a potential energy function, where the second indicator of the second conformation information characterizes binding energy of the complex conformation described by the second conformation information. In some embodiments, the second indicator of the second conformation information is the first quality indicator of the candidate ligand information. Alternatively, when there are at least two pieces of second conformation information, a median or a mean value of the second indicators may be determined based on the second indicators of the respective second conformation information, so as to obtain the first quality indicator of the candidate ligand information.

In implementation C, the target receptor information and the candidate ligand information are inputted into a binding affinity prediction model to obtain a target binding affinity, the target binding affinity being configured for characterizing an acting force upon binding of a receptor described by the target receptor information and a ligand described by the candidate ligand information. A first quality indicator of the candidate ligand information is determined based on the target binding affinity.

In the embodiments of the present disclosure, the electronic device may train a binding affinity prediction model, and the training manner is not limited herein. The embodiments of the present disclosure do not limit a model structure, model parameters, and the like of the binding affinity prediction model.

The binding affinity prediction model is a deep learning model, which may learn a binding manner of a receptor and a ligand, and predict an acting force during binding. The target receptor information and the candidate ligand information are inputted into the binding affinity prediction model, and feature fusion processing is performed on the target receptor information and the candidate ligand information using the binding affinity prediction model to obtain a target feature for characterizing a complex conformation formed by binding the receptor described by the target receptor information and the ligand described by the candidate ligand information. A target binding affinity is determined based on the target feature, and an acting force during binding of the receptor described by the target receptor information and the ligand described by the candidate ligand information is characterized by the target binding affinity.

The target binding affinity may be used as the first quality indicator of the candidate ligand information, or the target binding affinity may be mapped into the first quality indicator of the candidate ligand information.

In practical applications, at least two of implementation A to implementation C may alternatively be combined. To be specific, the first quality indicator of the candidate ligand information is determined based on at least two of the first indicator of the first conformation information, the second indicator of the second conformation information, the target binding affinity, and the like. The first quality indicator of the candidate ligand information may alternatively be determined in other manners. Details are not repeated herein.

The first quality indicator of the candidate ligand information is configured for characterizing the quality of the candidate ligand information. In some embodiments, a larger first quality indicator of the candidate ligand information indicates a higher quality of the candidate ligand information. If the first quality indicator of the candidate ligand information is not less than an indicator threshold, it is determined that the first quality indicator satisfies a first indicator condition. If the first quality indicator of the candidate ligand information is less than the indicator threshold, it is determined that the first quality indicator does not satisfy the first indicator condition. The indicator threshold may be a value determined according to manual experience, or a value adaptively adjusted according to actual situations. If the first quality indicator satisfies the first indicator condition, the candidate ligand information corresponding to the first quality indicator is the target ligand information.

Operation 2043: Train the reference generation model based on the sample receptor information, the sample ligand information, the target receptor information, and the target ligand information to obtain the ligand information generation model.

In the embodiments of the present disclosure, the ligand described by the target ligand information is of high quality, and the binding affinity between the ligand and the receptor described by the target receptor information is relatively high. The target receptor information and the target ligand information may be used as a high-quality data set D_{artificial}, and the high-quality data set and an original data set Dₙₐₜᵢᵥₑ including the sample receptor information and the sample ligand information are combined to obtain a training data set D=Dₙₐₜᵢᵥₑ+D_{artificial}. The reference generation model is trained using the training data set to obtain a ligand information generation model.

In some embodiments, the target receptor information and the sample receptor information in the training data set are collectively referred to as training receptor information, and the target ligand information and the sample ligand information in the training data set are collectively referred to as training ligand information. The reference noise data is denoised using the reference generation model based on the training receptor information to obtain first ligand information, and a third loss for representing a difference between the first ligand information and the training ligand information is determined. The reference generation model is trained based on the third loss to obtain the ligand information generation model. For the training manner, refer to the descriptions of operation 202 to operation 204. Implementation principles of the two operations are similar. Details are not repeated herein.

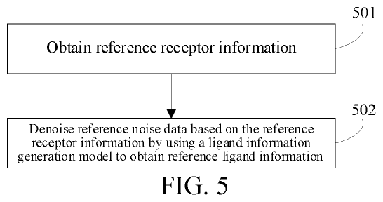

In the embodiments of the present disclosure, the target ligand information is determined using the reference generation model based on the target receptor information, which enriches the training data set of the model, and makes the training data of the model diverse and highly accurate. The training data set continues training the model, which can improve the accuracy and generalization ability of the model. The trained ligand information generation model is configured to generate reference ligand information based on reference receptor information. For details, refer to the following description related to FIG. 5. Details are not repeated herein.

FIG. 4 is a schematic diagram of training of a ligand information generation model according to an embodiment of the present disclosure. In the embodiments of the present disclosure, a sample information pair may be obtained, where the sample information pair includes sample receptor information and sample ligand information. A neural network model is first trained based on the sample information pair to obtain a reference generation model. Then, receptor database 1 and receptor database 2 are obtained, where receptor database 1 and receptor database 2 include at least two pieces of receptor information, and target receptor information is screened from the at least two pieces of receptor information.

Next, reference noise data is denoised using the reference generation model based on the target receptor information to obtain candidate ligand information. This process may be performed at least twice to obtain at least two pieces of candidate ligand information. In other words, generation is performed at least twice using the reference generation model based on the target receptor information and the reference noise data, to obtain at least two pieces of candidate ligand information.

For each piece of candidate ligand information, the candidate ligand information is scored based on the target receptor information to obtain a score of the candidate ligand information, where the score of the candidate ligand information corresponds to the first quality indicator of the candidate ligand information mentioned above. If the score is greater than a threshold, the candidate ligand information is used as target ligand information. If the score is not greater than the threshold, the candidate ligand information is discarded.

In this manner, the target receptor information and the target ligand information may be determined. The target receptor information and the target ligand information may be regarded as a target information pair. The reference generation model is further trained based on the target information pair and the sample information pair to obtain a ligand information generation model, and reference ligand information is generated using the ligand information generation model.

Information (including but not limited to user equipment information, user personal information, and the like), data (including but not limited to data for analysis, data for storage, data for display, and the like), and signals involved in the present disclosure are all authorized by users or fully authorized by all parties, and collection, use, and processing of relevant data need to comply with relevant laws, regulations, and standards of relevant regions. For example, the sample ligand information and the sample receptor information involved in the present disclosure are obtained with full authorization.

According to the method, reference noise data is first denoised based on sample receptor information using a neural network model to obtain predicted ligand information, a first loss is then determined based on the sample ligand information and the predicted ligand information, and the neural network model is trained based on the first loss, so that the neural network model is optimized in a direction to make the predicted ligand information close to the sample ligand information, thereby enabling the model to accurately output ligand information. In addition, since a binding affinity between a ligand described by the sample ligand information and a receptor described by the sample receptor information is not less than a preset affinity, a trained ligand information generation model can generate reference ligand information based on reference receptor information, and a binding affinity between a ligand described by the reference ligand information and a receptor described by the reference receptor information is high.

FIG. 5 is a flowchart of a method for generating ligand information according to an embodiment of the present disclosure. For ease of description, the terminal device 101 or the server 102 for performing the method for generating ligand information in embodiments of the present disclosure is referred to as an electronic device. To be specific, the method may be performed by the electronic device. As shown in FIG. 5, the method includes the following operations.

Operation 501: Obtain reference receptor information.

The embodiments of the present disclosure do not limit the manner of obtaining the reference receptor information. By way of example, the electronic device may obtain reference receptor information inputted by a user. Alternatively, the electronic device may acquire a target polypeptide data set from the Internet. The target polypeptide data set includes at least two target polypeptide pairs. One target polypeptide pair includes target information. The electronic device maps the target information into text features using an embedding transformation function to obtain the reference receptor information. The implementation of operation 501 is similar to that of operation 201, which may refer to the description of operation 201. Details are not repeated herein.

Operation 502: Denoise reference noise data based on the reference receptor information using a ligand information generation model to obtain reference ligand information.

The ligand information generation model is trained according to the method related to FIG. 2. The reference noise data may be inputted into the ligand information generation model. I addition, the reference receptor information may be inputted into the ligand information generation model. In one embodiment, the reference noise data and the reference receptor information are concatenated to obtain concatenated information, and the concatenated information is inputted into the ligand information generation model. The ligand information generation model is a denoising network model, and is configured to denoise the reference noise data based on the reference receptor information to obtain reference ligand information. The reference ligand information is feature data for characterizing a ligand obtained by prediction through the neural network model. The implementation of operation 502 is similar to that of operation 202, which may refer to the description of operation 202. Details are not repeated herein.

In one embodiment, operation 502 includes: denoising the reference noise data based on the reference receptor information using the ligand information generation model to obtain at least two pieces of alternative ligand information; determining a second quality indicator for characterizing quality of the respective alternative ligand information; and selecting, from the at least two pieces of alternative ligand information, the reference ligand information in which the second quality indicator satisfies a second indicator condition.

In the embodiments of the present disclosure, for the content of generating alternative ligand information using the ligand information generation model, refer to the above description related to the generation process of candidate ligand information. Details are not repeated herein. Next, a second quality indicator of the alternative ligand information may be determined according to the determination principle of the first quality indicator of the candidate ligand information. For details, refer to the above descriptions related to implementation A to implementation C. Details are not repeated herein.

The second quality indicator of the alternative ligand information is configured for characterizing the quality of the alternative ligand information. In some embodiments, a larger second quality indicator of the alternative ligand information indicates a higher quality of the alternative ligand information. If the second quality indicator of the alternative ligand information is not less than an indicator threshold, it is determined that the second quality indicator satisfies a second indicator condition. If the second quality indicator of the alternative ligand information is less than the indicator threshold, it is determined that the second quality indicator does not satisfy the second indicator condition. The indicator threshold may be a value determined according to manual experience, or a value adaptively adjusted according to actual situations. If the second quality indicator satisfies the second indicator condition, the alternative ligand information corresponding to the second quality indicator is the reference ligand information.

In practical applications, the ligand information generation model in the embodiments of the present disclosure may be concatenated with other models to implement corresponding functions based on these models. For example, a three-dimensional structure prediction model is concatenated following the ligand information generation model. After the ligand information generation model generates reference ligand information, the reference ligand information is inputted into the three-dimensional structure prediction model, and a three-dimensional structure of a ligand described by the reference ligand information after the ligand binds to a receptor is predicted using the three-dimensional structure prediction model. For another example, a complex conformation prediction model is concatenated following the ligand information generation model. After the ligand information generation model generates reference ligand information, the reference ligand information is inputted into the complex conformation prediction model, and a structure formed by the binding of the ligand described by the reference ligand information to the receptor is predicted using the complex conformation prediction model.

Information (including but not limited to user equipment information, user personal information, and the like), data (including but not limited to data for analysis, data for storage, data for display, and the like), and signals involved in the present disclosure are all authorized by users or fully authorized by all parties, and collection, use, and processing of relevant data need to comply with relevant laws, regulations, and standards of relevant regions. For example, the reference receptor information involved in the present disclosure is obtained with full authorization.

The ligand information generation model in the foregoing method is trained using the related method in FIG. 2, and can generate reference ligand information based on reference receptor information, and a binding affinity between a ligand described by the reference ligand information and a receptor described by the reference receptor information is relatively high.

The method according to the embodiments of the present disclosure is described above from the perspective of method operations, and the method according to the embodiments of the present disclosure is systematically described below. The method according to the embodiments of the present disclosure may be applied to various scenarios such as drug design and targeted therapy. In these scenarios, a ligand information generation model is trained to generate ligand information based on receptor information using the ligand information generation model, so as to implement a specific generation task. A receptor and a ligand include, but are not limited to: an antigen and an antibody, a protein and a protein, a protein and a polypeptide sequence, a protein and a small molecule, and the like. The method according to the embodiments of the present disclosure is described below using an example in which the receptor is a protein and the ligand is a polypeptide sequence.

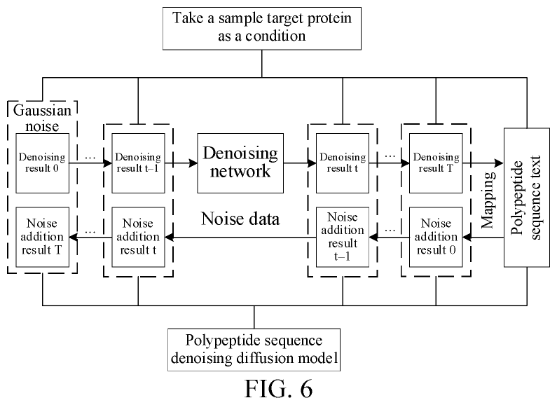

FIG. 6 is a schematic diagram of training of a polypeptide sequence denoising diffusion model using a sample target protein as a condition according to an embodiment of the present disclosure. The training process includes a forward diffusion process and a reverse diffusion process.

The forward diffusion process is a process of gradually adding noise to an embedding feature of a polypeptide sequence text. In the embodiments of the present disclosure, the polypeptide sequence text includes at least two amino acid words. The embedding feature of the polypeptide sequence text is obtained by mapping the respective amino acid words into corresponding vector codes, and the embedding feature of the polypeptide sequence text corresponds to the sample ligand information mentioned above.

The embedding feature of the polypeptide sequence text is non-noised feature data, which may be referred to as noise addition result 0. Noise data is determined in any manner, and noise is added to noise addition result 0 based on the noise data to obtain noise addition result 1. Noise data is determined in any manner, and noise is added to noise addition result 1 based on the noise data to obtain noise addition result 2. By analogy, noise addition is gradually performed T times on noise addition result 0 to obtain noise addition result T. Noise addition result Tis Gaussian noise, and noise addition result T corresponds to the reference noise data mentioned above.

The reverse diffusion process is a process of gradually denoising the Gaussian noise. The Gaussian noise is non-denoised data, which may be referred to as denoising result 0. Denoising result 0 is denoised using a denoising network based on a sample target protein to obtain denoising result 1. Denoising result 1 is denoised using a denoising network based on a sample target protein to obtain denoising result 2. By analogy, denoising is gradually performed T times on denoising result 0 based on the sample target protein to obtain denoising result T. Denoising result T is the embedding feature of the polypeptide sequence text, and the embedding feature of the polypeptide sequence text may be mapped into the polypeptide sequence text.

A neural network model is trained based on the embedding feature of the polypeptide sequence text in the noise addition process and the embedding feature of the polypeptide sequence text obtained by denoising, so as to obtain a polypeptide sequence denoising diffusion model using the sample target protein as a condition, and the model corresponds to the ligand information generation model mentioned above.

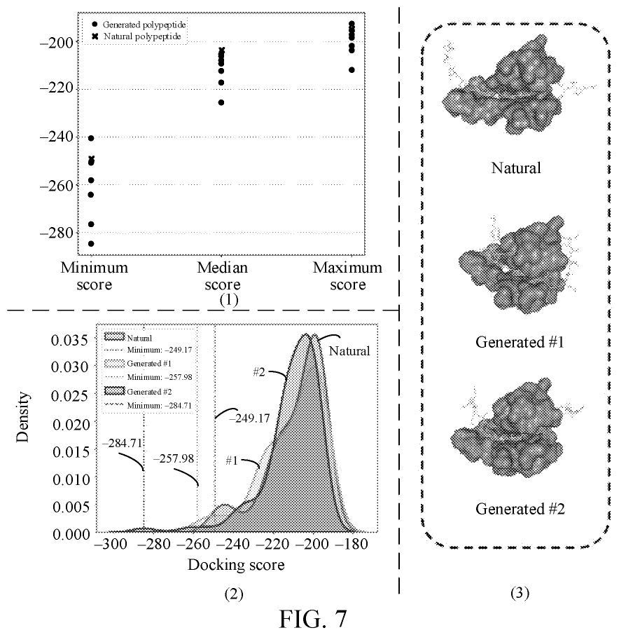

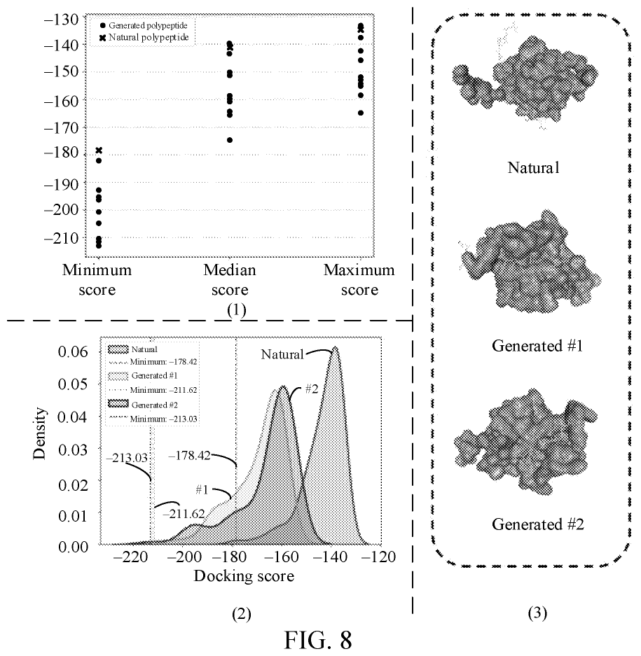

The polypeptide sequence generation model obtained in the embodiments of the present disclosure can generate a polypeptide sequence based on the text of a target protein. In some embodiments, the target protein is an aspartate 1-decarboxylase alpha chain. A natural polypeptide and generated polypeptides corresponding to the target protein are shown in FIG. 7. The target protein is a Bcl-w protein. A natural polypeptide and generated polypeptides corresponding to the target protein are shown in FIG. 8. It can be seen from (1) to (3) in FIGS. 7 and (1) to (3) in FIG. 8 that, in most cases, the polypeptides generated by the polypeptide sequence generation model can achieve a lower molecular docking score than the natural polypeptide, which indicates that the generated polypeptides are more stably bound to the target protein and have a higher binding affinity.

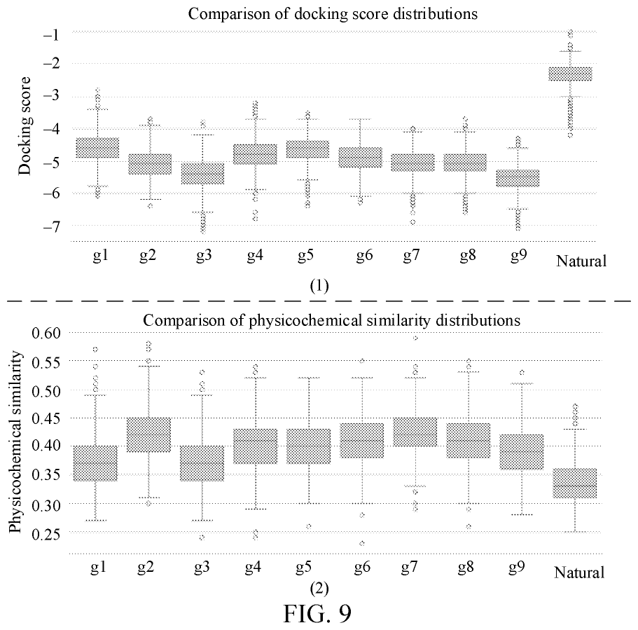

Polypeptides (i.e., g1 to g9) are generated by the polypeptide sequence generation model based on a glucagon-like peptide 1 receptor (GLP-1R). The generated polypeptides are compared with the natural polypeptide, so as to obtain a schematic diagram of distribution comparison of docking scores as shown in (1) in FIG. 9 and physicochemical similarities as shown in (2) in FIG. 9. As shown in FIG. 9, compared with the natural polypeptide, the generated polypeptides can achieve lower docking scores (Vina Score) and higher physicochemical similarities (PC Score), which indicates that the generated polypeptides have a higher binding affinity to the target protein and are closer to existing polypeptides in physicochemical properties.

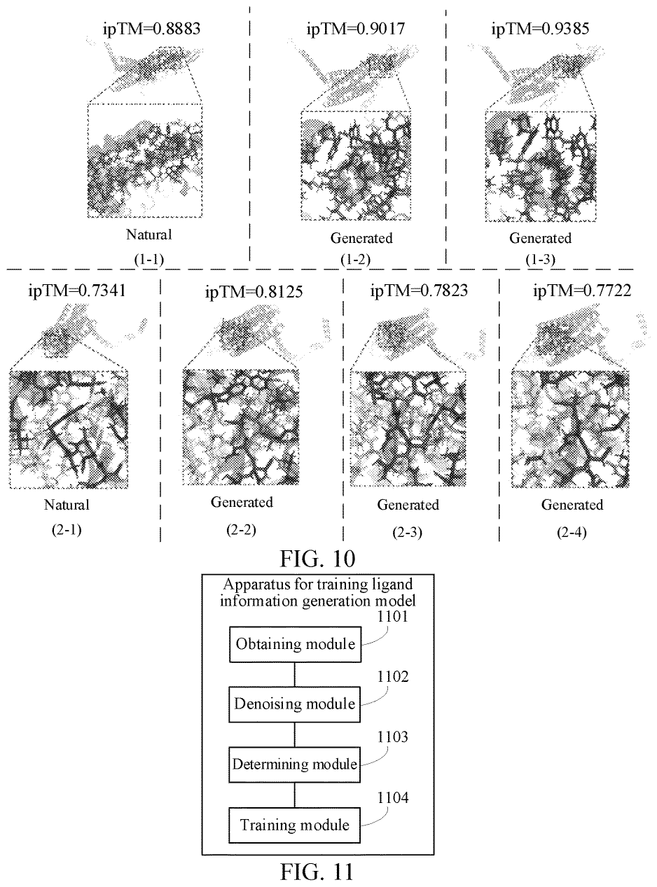

In addition, on the one hand, polypeptides are generated using the polypeptide sequence generation model based on GLP-1R, and an interface predicted Tm-score (ipTM) confidence of the natural polypeptide and an ipTM confidence of the generated polypeptides are obtained using a tool, so as to obtain results as shown in (1-1) to (1-3) in FIG. 10. It can be seen that the binding reliability between the generated polypeptides and the target protein is higher. On the other hand, polypeptides are generated using the polypeptide sequence generation model based on somatostatin receptor 5 (SSTR5), and ipTM confidences of the natural polypeptide and the generated polypeptides are obtained using a tool, so as to obtain results as shown in (2-1) to (2-4) in FIG. 10. The results show that: the embodiments of the present disclosure can generate, with high quality and high speed, a polypeptide sequence having a potential high affinity with a target protein, which is of great significance for drug design in personalized medicine, cancer targeted therapy, and the like.

FIG. 11 shows a schematic structural diagram of an apparatus for training a ligand information generation model according to an embodiment of the present disclosure. The apparatus includes:
an obtaining module 1101, configured to obtain sample receptor information and sample ligand information, a binding affinity between a ligand described by the sample ligand information and a receptor described by the sample receptor information being not less than a set affinity;
a denoising module 1102, configured to denoise reference noise data based on the sample receptor information using a to-be-trained neural network model to obtain predicted ligand information;
a determining module 1103, configured to determine a first loss for characterizing
a difference between the sample ligand information and the predicted ligand information; and
a training module 1104, configured to train the neural network model based on the first loss to obtain a ligand information generation model, the ligand information generation model being configured to generate reference ligand information based on reference receptor information.

In one embodiment, the neural network model includes at least two denoising networks.

The denoising module 1102 is configured to: denoise, for a first denoising network, the reference noise data based on the sample receptor information using the first denoising network to obtain a denoising result of the first denoising network; denoise, for a non-first denoising network, a denoising result of a previous denoising network of the non-first denoising network based on the sample receptor information using the non-first denoising network to obtain a denoising result of the non-first denoising network; and determine the predicted ligand information based on a denoising result of a last denoising network.

In one embodiment, the first denoising network includes an attention network and a feed-forward network.

The denoising module 1102 is configured to: perform attention processing on the sample receptor information and the reference noise data using the attention network to obtain an attention processing result; and determine a denoising result of the first denoising network based on the attention processing result using the feed-forward network.

In one embodiment, the sample receptor information includes at least two pieces of receptor component information, and the reference noise data includes at least one piece of first sub-data.

The denoising module 1102 is configured to: perform, for any first sub-data, attention processing on the any first sub-data and content information using the attention network to obtain a processing result of the content information, and determine second sub-data corresponding to the any first sub-data based on the processing result of the content information, the content information including the at least two pieces of receptor component information and the at least one piece of first sub-data; and determine the attention processing result based on the second sub-data corresponding to the respective first sub-data.

In one embodiment, the denoising module 1102 is configured to: perform mapping on the attention processing result using the feed-forward network to obtain a mapping result; and perform mapping on the mapping result or a set feature using the feed-forward network to obtain the denoising result of the first denoising network.

In one embodiment, the denoising result of the last denoising network includes at least two predicted sub-features.

The denoising module 1102 is configured to: screen, for any predicted sub-feature, at least two pieces of target ligand component information from at least two pieces of candidate ligand component information based on feature similarity between the any predicted sub-feature and the at least two pieces of candidate ligand component information, and sample from the at least two pieces of target ligand component information to obtain ligand component information corresponding to the any predicted sub-feature; and determine the predicted ligand information based on the ligand component information corresponding to the respective predicted sub-features.

In one embodiment, the apparatus further includes:
a noise addition module, configured to add noise to the sample ligand information to obtain the reference noise data.

In one embodiment, noise addition is performed multiple times.

The noise addition module is configured to: perform, for a first noise addition, the first noise addition on the sample ligand information to obtain a noise addition result of the first noise addition; and perform, for a non-first noise addition, the non-first noise addition on a noise addition result of a previous noise addition of the non-first noise addition to obtain a noise addition result of the non-first noise addition, a noise addition result of a last noise addition being the reference noise data.

In one embodiment, the noise addition and the denoising are both performed T times, T being a positive integer.

The training module 1104 is configured to: determine, for an i^{th} denoising, a second loss corresponding to the i^{th} denoising based on noise data removed during the i^{th} denoising and noise data added during a (T+1−i)^{th} noise addition, i being a positive integer from 1 to T; and train the neural network model based on the first loss and the second loss corresponding to each denoising to obtain the ligand information generation model.

In one embodiment, the training module 1104 is configured to: train the neural network model based on the first loss to obtain a reference generation model; denoise the reference noise data based on target receptor information using the reference generation model to obtain target ligand information; and train the reference generation model based on the sample receptor information, the sample ligand information, the target receptor information, and the target ligand information to obtain the ligand information generation model.

In one embodiment, the training module 1104 is configured to: denoise the reference noise data based on target receptor information using the reference generation model to obtain candidate ligand information; and determine, if a first quality indicator for characterizing quality of the candidate ligand information satisfies a first indicator condition, the candidate ligand information as the target ligand information.

In one embodiment, the determining module 1103 is further configured to: determine at least two pieces of first conformation information based on the target receptor information and the candidate ligand information, the first conformation information being configured for describing a complex conformation formed after binding a receptor described by the target receptor information and a ligand described by the candidate ligand information; determine, for any first conformation information, a first indicator of the any first conformation information, the first indicator being configured for characterizing binding energy of a complex conformation described by the any first conformation information; and determine a first quality indicator of the candidate ligand information based on the first indicator of the respective first conformation information.

In one embodiment, the apparatus further includes:
a selection module, configured to select reference conformation information from a database, an alignment result between ligand information included in the reference conformation information and the candidate ligand information satisfying an alignment condition, and an alignment result between receptor information included in the reference conformation information and the target receptor information satisfying the alignment condition.

The determining module 1103 is further configured to: determine second conformation information based on the reference conformation information, the target receptor information, and the candidate ligand information, the second conformation information being configured for describing a complex conformation formed after binding a receptor described by the target receptor information and a ligand described by the candidate ligand information; and determine a first quality indicator of the candidate ligand information based on the second conformation information.

In one embodiment, the determining module 1103 is further configured to: input the target receptor information and the candidate ligand information into a binding affinity prediction model to obtain a target binding affinity, the target binding affinity being configured for characterizing an acting force upon binding of a receptor described by the target receptor information and a ligand described by the candidate ligand information; and determine a first quality indicator of the candidate ligand information based on the target binding affinity.

The apparatus first denoises reference noise data based on sample receptor information using a neural network model to obtain predicted ligand information, then determines a first loss based on the sample ligand information and the predicted ligand information, and trains the neural network model based on the first loss, so that the neural network model is optimized in a direction to make the predicted ligand information close to the sample ligand information, thereby enabling the model to accurately output ligand information. In addition, since a binding affinity between a ligand described by the sample ligand information and a receptor described by the sample receptor information is not less than a preset affinity, a trained ligand information generation model can generate reference ligand information based on reference receptor information, and a binding affinity between a ligand described by the reference ligand information and a receptor described by the reference receptor information is high.

When the apparatus provided in FIG. 11 implements functions of the apparatus, the division of the foregoing functional modules is merely used as an example for description. In practical application, the functions may be allocated to and completed by different functional modules according to requirements. To be specific, an internal structure of a device is divided into different functional modules to complete all or some of the functions described above. In addition, the apparatus provided in the foregoing embodiment belongs to the same conception as the embodiment of the method. For a specific implementation process thereof, refer to the method embodiment. Details are not repeated herein.

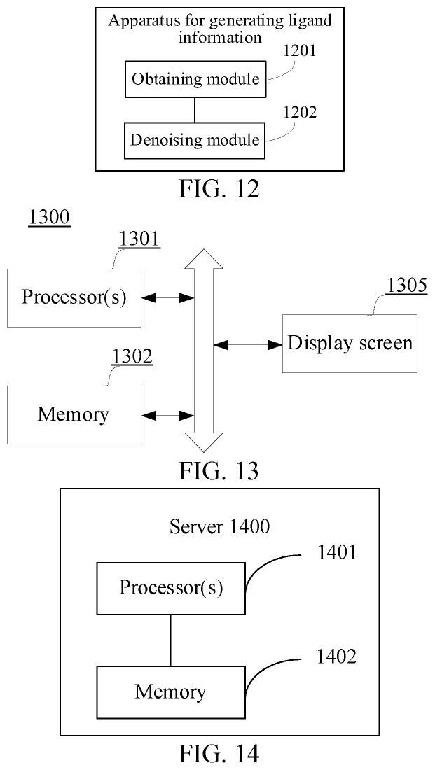

FIG. 12 shows a schematic structural diagram of an apparatus for generating ligand information according to an embodiment of the present disclosure. The apparatus includes:
an obtaining module 1201, configured to obtain reference receptor information; and
a denoising module 1202, configured to denoise reference noise data based on the reference receptor information using a ligand information generation model to obtain reference ligand information, the ligand information generation model being trained using the method according to the first aspect.

In one embodiment, the denoising module 1202 is configured to: denoise the reference noise data based on the reference receptor information using the ligand information generation model to obtain at least two pieces of alternative ligand information; determine a second quality indicator for characterizing quality of the respective alternative ligand information; and select, from the at least two pieces of alternative ligand information, the reference ligand information in which the second quality indicator satisfies a second indicator condition.

The ligand information generation model in the foregoing apparatus is trained using the related method in FIG. 2, and can generate reference ligand information based on reference receptor information, and a binding affinity between a ligand described by the reference ligand information and a receptor described by the reference receptor information is relatively high.

When the apparatus provided in FIG. 12 implements functions of the apparatus, the division of the foregoing functional modules is merely used as an example for description. In practical application, the functions may be allocated to and completed by different functional modules according to requirements. To be specific, an internal structure of a device is divided into different functional modules to complete all or some of the functions described above. In addition, the apparatus provided in the foregoing embodiment belongs to the same conception as the embodiment of the method. For a specific implementation process thereof, refer to the method embodiment. Details are not repeated herein.

FIG. 13 shows a structural block diagram of a terminal device 1300 according to an exemplary embodiment of the present disclosure. The terminal device 1300 includes a processor 1301 and a memory 1302.

The processor 1301 may include one or more processing cores, for example, a 4-core processor or an 8-core processor. The processor 1301 may be implemented in at least one hardware form of digital signal processing (DSP), a field-programmable gate array (FPGA), and a programmable logic array (PLA). The processor 1301 may alternatively include a main processor and a coprocessor. The main processor is a processor for processing data in an awake state, and is also referred to as a central processing unit (CPU). The coprocessor is a low power consumption processor for processing the data in a standby state. In some embodiments, the processor 1301 may be integrated with a graphics processing unit (GPU). The GPU is configured to render and draw content that needs to be displayed on a display screen. In some embodiments, the processor 1301 may further include an artificial intelligence (AI) processor. The AI processor is configured to perform computing operations related to machine learning.

The memory 1302 may include one or more computer-readable storage media. The computer-readable storage medium may be non-transient. The memory 1302 may further include a high-speed random access memory, and a non-volatile memory, for example, one or more disk storage devices and flash storage devices. In some embodiments, the non-transient computer-readable storage medium in the memory 1302 is configured to store at least one computer program. The at least one computer program is executed by the processor 1301 to implement the method for training a ligand information generation model and the method for generating ligand information provided in the method embodiments of the present disclosure.

In some embodiments, the terminal device 1300 may alternatively include a display screen 1305. The display screen 1305 is configured to display a user interface (UI). The UI may include graphics, text, icons, videos, and any combination thereof. By way of example, the UI includes any one or more of receptor information and ligand information mentioned above.

A person skilled in the art may understand that the structure shown in FIG. 13 constitutes no limitation on the terminal device 1300. The terminal device may include more or fewer components than those shown in the figure, or some components may be combined, or a different component deployment may be used.

FIG. 14 is a schematic structural diagram of a server according to an embodiment of the present disclosure. The server 1400 may vary considerably due to different configurations or performances, and may include one or more processors 1401 and one or more memories 1402. The one or more memories 1402 have at least one computer program stored therein. The at least one computer program is loaded and executed by the one or more processors 1401 to implement the method for training a ligand information generation model or the method for generating ligand information provided by the respective method embodiments. By way of example, the processor 1401 is a CPU. In particular, the server 1400 may have a wired or wireless network interface, a keyboard, an input/output interface, and other components, to facilitate input/output. The server 1400 may alternatively include other components for implementing device functions. Details are not described herein.

In an exemplary embodiment, a non-volatile computer-readable storage medium is further provided. The non-volatile computer-readable storage medium has at least one computer program stored therein, and the at least one computer program is loaded and executed by a processor, to cause an electronic device to implement the method for training a ligand information generation model or the method for generating ligand information according to any one of the foregoing aspects.

In some embodiments, the non-volatile computer-readable storage medium may be a read-only memory (ROM), a random access memory (RAM), a compact disc read-only memory (CD-ROM), a magnetic tape, a floppy disk, an optical data storage device, or the like.

In an exemplary embodiment, a computer program is further provided. There is at least one computer program, and the at least one computer program is loaded and executed by a processor, to cause an electronic device to implement the method for training a ligand information generation model or the method for generating ligand information according to any one of the foregoing aspects.

In an exemplary embodiment, a computer program product is further provided. The computer program product has at least one computer program stored therein, and the at least one computer program is loaded and executed by a processor, to cause an electronic device to implement the method for training a ligand information generation model or the method for generating ligand information according to any one of the foregoing aspects.

The term module (and other similar terms such as submodule, unit, subunit, etc.) in this disclosure may refer to a software module, a hardware module, or a combination thereof. A software module (e.g., computer program) may be developed using a computer programming language. A hardware module may be implemented using processing circuitry and/or memory. Each module can be implemented using one or more processors (or processors and memory). Likewise, a processor (or processors and memory) can be used to implement one or more modules. Moreover, each module can be part of an overall module that includes the functionalities of the module.

“Plurality of” mentioned in the specification means two or more. “And/or” describes an association relationship for describing associated objects and represents that three relationships may exist. For example, A and/or B may represent the following three cases: Only A exists, both A and B exist, and only B exists. The character “/” generally indicates an “or” relationship between the associated objects. At least one mentioned in this specification means one or more. Similarly, at least two means two or more, and at least three means three or more.

The sequence numbers of the foregoing embodiments of the present disclosure are for description purposes only and do not represent the superiority or inferiority of the embodiments.

According to the technical solutions provided in the present disclosure, reference noise data is first denoised based on sample receptor information using a neural network model to obtain predicted ligand information, a first loss is then determined based on the sample ligand information and the predicted ligand information, and the neural network model is trained based on the first loss, so that the neural network model is optimized in a direction to make the predicted ligand information close to the sample ligand information, thereby enabling the model to accurately output ligand information. In addition, since a binding affinity between a ligand described by the sample ligand information and a receptor described by the sample receptor information is not less than a preset affinity, a trained ligand information generation model can generate reference ligand information based on reference receptor information, and a binding affinity between a ligand described by the reference ligand information and a receptor described by the reference receptor information is high.

The foregoing descriptions are merely exemplary embodiments of the present disclosure, but are not intended to limit the present disclosure. Any modification, equivalent replacement, or improvement made within the principle of the present disclosure fall within the protection scope of the present disclosure.

What is claimed is:

1. A method for training a ligand information generation model, performed by an electronic device, the method comprising:
obtaining sample receptor information and sample ligand information, a binding affinity between a ligand described by the sample ligand information and a receptor described by the sample receptor information being not less than a set affinity;
denoising reference noise data based on the sample receptor information using a neural network model undergoing training to obtain predicted ligand information;
determining a first loss for characterizing a difference between the sample ligand information and the predicted ligand information; and
training the neural network model based on the first loss to obtain a ligand information generation model, the ligand information generation model being configured to generate reference ligand information based on reference receptor information.

2. The method according to claim 1, wherein the neural network model comprises at least two denoising networks, and denoising the reference noise data based on the sample receptor information using the neural network model undergoing training to obtain predicted ligand information comprises:
denoising, for a first denoising network of the at least two denoising networks, the reference noise data based on the sample receptor information using the first denoising network to obtain a denoising result of the first denoising network;
denoising, for a non-first denoising network of the at least two denoising networks, a denoising result of a previous denoising network of the non-first denoising network based on the sample receptor information using the non-first denoising network to obtain a denoising result of the non-first denoising network; and
determining the predicted ligand information based on a denoising result of a last denoising network.

3. The method according to claim 2, wherein the first denoising network comprises an attention network and a feed-forward network, and denoising, for the first denoising network of the at least two denoising networks, the reference noise data based on the sample receptor information using the first denoising network to obtain the denoising result of the first denoising network comprises:
performing attention processing on the sample receptor information and the reference noise data using the attention network to obtain an attention processing result; and
determining a denoising result of the first denoising network based on the attention processing result using the feed-forward network.

4. The method according to claim 3, wherein the sample receptor information comprises at least two receptor component information pieces, the reference noise data comprises at least one first sub-data, and performing the attention processing on the sample receptor information and the reference noise data using the attention network to obtain the attention processing result comprises:
performing, for any first sub-data, attention processing on the any first sub-data and content information using the attention network to obtain a processing result of the content information, and determining second sub-data corresponding to the any first sub-data based on the processing result of the content information, the content information comprising the at least two receptor component information pieces and the at least one first sub-data; and
determining the attention processing result based on the second sub-data corresponding to the respective first sub-data.

5. The method according to claim 3, wherein determining the denoising result of the first denoising network based on the attention processing result using the feed-forward network comprises:
performing mapping on the attention processing result using the feed-forward network to obtain a mapping result; and
performing mapping on the mapping result or a set feature using the feed-forward network to obtain the denoising result of the first denoising network.

6. The method according to claim 2, wherein the denoising result of the last denoising network comprises at least two predicted sub-features, and determining the predicted ligand information based on the denoising result of the last denoising network comprises:
screening, for any predicted sub-feature, at least two target ligand component information pieces from at least two candidate ligand component information pieces based on feature similarity between the any predicted sub-feature and the at least two candidate ligand component information pieces, and sampling from the at least two target ligand component information pieces to obtain ligand component information corresponding to the any predicted sub-feature; and
determining the predicted ligand information based on the ligand component information corresponding to the respective predicted sub-features.

7. The method according to claim 1, further comprising:
adding noise to the sample ligand information to obtain the reference noise data.

8. The method according to claim 7, wherein noise addition is performed multiple times, and adding the noise to the sample ligand information to obtain the reference noise data comprises:
performing, for a first noise addition, the first noise addition on the sample ligand information to obtain a noise addition result of the first noise addition; and
performing, for a non-first noise addition, the non-first noise addition on a noise addition result of a previous noise addition of the non-first noise addition to obtain a noise addition result of the non-first noise addition, a noise addition result of a last noise addition being the reference noise data.

9. The method according to claim 8, wherein the noise addition and the denoising are both performed T times, T being a positive integer, and training the neural network model based on the first loss to obtain the ligand information generation model comprises:
determining, for an i^{th} denoising, a second loss corresponding to the i^{th} denoising based on noise data removed during the i^{th} denoising and noise data added during a (T+1−i)^{th} noise addition, i being a positive integer from 1 to T; and
training the neural network model based on the first loss and the second loss corresponding to each denoising to obtain the ligand information generation model.

10. The method according to claim 1, wherein the training the neural network model based on the first loss to obtain the ligand information generation model comprises:
training the neural network model based on the first loss to obtain a reference generation model;
denoising the reference noise data based on target receptor information using the reference generation model to obtain target ligand information; and
training the reference generation model based on the sample receptor information, the sample ligand information, the target receptor information, and the target ligand information to obtain the ligand information generation model.

11. The method according to claim 10, wherein denoising the reference noise data based on the target receptor information using the reference generation model to obtain the target ligand information comprises:
denoising the reference noise data based on target receptor information using the reference generation model to obtain candidate ligand information; and
determining, if a first quality indicator for characterizing quality of the candidate ligand information satisfies a first indicator condition, the candidate ligand information as the target ligand information.

12. The method according to claim 11, further comprising:
determining at least two first conformation information pieces based on the target receptor information and the candidate ligand information, the first conformation information being configured for describing a complex conformation formed after binding a receptor described by the target receptor information and a ligand described by the candidate ligand information;
determining, for any first conformation information, a first indicator of the any first conformation information, the first indicator being configured for characterizing binding energy of a complex conformation described by the any first conformation information; and
determining a first quality indicator of the candidate ligand information based on the first indicator of the respective first conformation information.

13. The method according to claim 11, further comprising:
selecting reference conformation information from a database, an alignment result between ligand information comprised in the reference conformation information and the candidate ligand information satisfying an alignment condition, and an alignment result between receptor information comprised in the reference conformation information and the target receptor information satisfying the alignment condition;
determining second conformation information based on the reference conformation information, the target receptor information, and the candidate ligand information, the second conformation information being configured for describing a complex conformation formed after binding a receptor described by the target receptor information and a ligand described by the candidate ligand information; and
determining a first quality indicator of the candidate ligand information based on the second conformation information.

14. The method according to claim 11, further comprising:
inputting the target receptor information and the candidate ligand information into a binding affinity prediction model to obtain a target binding affinity, the target binding affinity being configured for characterizing an acting force upon binding of a receptor described by the target receptor information and a ligand described by the candidate ligand information; and
determining a first quality indicator of the candidate ligand information based on the target binding affinity.

15. The method according to claim 1, further comprising:
generating ligand information, by performing:
obtaining reference receptor information; and
denoising reference noise data based on the reference receptor information using the ligand information generation model to obtain reference ligand information.

16. An electronic device, comprising one or more processors and a memory containing at least one computer program that, when being executed, causes the one or more processors to perform:
obtaining sample receptor information and sample ligand information, a binding affinity between a ligand described by the sample ligand information and a receptor described by the sample receptor information being not less than a set affinity;
denoising reference noise data based on the sample receptor information using a neural network model undergoing training to obtain predicted ligand information;
determining a first loss for characterizing a difference between the sample ligand information and the predicted ligand information; and
training the neural network model based on the first loss to obtain a ligand information generation model, the ligand information generation model being configured to generate reference ligand information based on reference receptor information.

17. The device according to claim 16, wherein the neural network model comprises at least two denoising networks, and the one or more processors are further configured to perform:
denoising, for a first denoising network of the at least two denoising networks, the reference noise data based on the sample receptor information using the first denoising network to obtain a denoising result of the first denoising network;
denoising, for a non-first denoising network of the at least two denoising networks, a denoising result of a previous denoising network of the non-first denoising network based on the sample receptor information using the non-first denoising network to obtain a denoising result of the non-first denoising network; and
determining the predicted ligand information based on a denoising result of a last denoising network.

18. The device according to claim 17, wherein the first denoising network comprises an attention network and a feed-forward network, and the one or more processors are further configured to perform:
performing attention processing on the sample receptor information and the reference noise data using the attention network to obtain an attention processing result; and
determining a denoising result of the first denoising network based on the attention processing result using the feed-forward network.

19. The device according to claim 18, wherein the sample receptor information comprises at least two receptor component information pieces, the reference noise data comprises at least one first sub-data, and the one or more processors are further configured to perform:
performing, for any first sub-data, attention processing on the any first sub-data and content information using the attention network to obtain a processing result of the content information, and determining second sub-data corresponding to the any first sub-data based on the processing result of the content information, the content information comprising the at least two receptor component information pieces and the at least one first sub-data; and
determining the attention processing result based on the second sub-data corresponding to the respective first sub-data.

20. A non-transitory computer-readable storage medium containing at least one computer program that, when being executed, causes the one or more processors to perform:
obtaining sample receptor information and sample ligand information, a binding affinity between a ligand described by the sample ligand information and a receptor described by the sample receptor information being not less than a set affinity;
denoising reference noise data based on the sample receptor information using a neural network model undergoing training to obtain predicted ligand information;
determining a first loss for characterizing a difference between the sample ligand information and the predicted ligand information; and
training the neural network model based on the first loss to obtain a ligand information generation model, the ligand information generation model being configured to generate reference ligand information based on reference receptor information.
